(19) United States
(12) Patent Application Publication
(10) Pub. No.: US 2025/0275908 A1
(43) Pub. Date: Sep. 4, 2025

(54) COSMETIC COMPOSITION FOR ACNE CARE

(71) Applicant: L'OREAL, Paris (FR)

(72) Inventors: Changlong Chen, Edison, NJ (US); Siliu TAN, Westfield, NJ (US); Christopher PANG, New York, NY (US); Mariko HASEBE, New York, NY (US)

(73) Assignee: L'OREAL, Paris (FR)

(21) Appl. No.: 18/591,930

(22) Filed: Feb. 29, 2024

(51) Int. Cl. A61K 8/96; A61K 8/365; A61K 8/37; A61K 8/60; A61Q 19/10

(52) U.S. Cl. CPC A61K 8/965; A61K 8/365; A61K 8/37; A61K 8/602; A61Q 19/10

(57) ABSTRACT

An acne care composition includes at least one surfactant comprising sodium cocoyl isethionate and optionally glyceryl stearate SE, at least one glycolipid, at least one beta hydroxy acid comprising salicylic acid or a derivative thereof, at least one clay optionally comprising kaolin, at least one cationic polymer optionally comprising chitosan, at least one thickener comprising one of carrageenan, xanthan gum, sclerotium gum, or a combination thereof, at least one cosmetically acceptable solvent comprising water, and comprising glycerin, and at least one additive selected from the group consisting of sodium hydroxide, niacinamide, lactic acid, cetyl alcohol, trisodium ethylenediamine disuccinate, ceramides, and combinations thereof. When applied to skin by rubbing, the composition demonstrates peak shear stress in a range from about 100 to about 1000 PA at shear rate in the range from 1 1/s-100 1/s, followed by a sharp drop in viscosity when shear stress is maintained.

### TECHNICAL FIELD

The present disclosure is directed to a skin care composition, in particular, an acne care composition.

### BACKGROUND

Current acne skincare routines often require application of acne care products which are left on the skin for a desired period of time to yield the benefit of the active ingredients. Such products can suffer from being difficult to spread, unreliable to retain without dripping on acne-prone areas and spots during treatment, and difficult removal when the application time is complete.

There is a need in the market for acne skincare compositions that have beneficial levels of actives provided in a base that allows ease of application to skin and reliable retention on the targeted areas until desired removal at which time the composition may be reliably removed.

The inventors provide here a product formulation in the form of an acne care composition with paste texture that is easy to spread under shear and has good structure without dripping after application and until removal.

### SUMMARY

In a first embodiment, the acne care composition comprises
a) at least one surfactant comprising sodium cocoyl isethionate;
b) at least one glycolipid;
c) at least one beta hydroxy acid;
d) at least one clay; and
e) at least one cosmetically acceptable solvent comprising water and glycerin,
   wherein the acne care composition demonstrates peak shear stress in a range from about 100 to about 1000 PA at shear rate in the range from 1 1/s-100 1/s, followed by a sharp drop in viscosity when shear stress is maintained.
   In some embodiments, the at least one glycolipid is chosen from rhamnolipids, sophorolipids, glucolipids, trehalolipids, cellobiose lipids, mannosylerythritol lipid, or a mixture thereof.

In some embodiments, the at least one glycolipid is chosen from rhamnolipids present from about 1% to about 4%, by weight, based on the weight of the acne care composition.

In some embodiments, the at least one beta hydroxy acid comprises salicylic acid or a derivative thereof present from about 0.1% to about 2%, by weight, based on the weight of the acne care composition.

In some embodiments, the at least one clay comprises kaolin present from about 5% to about 20%, by weight, based on the weight of the acne care composition.

In some embodiments, glycerin is present from about 10% to about 30%, by weight, based on the weight of the acne care composition.

In some embodiments, the acne care composition comprises at least one cationic polymer.

In some embodiments, the at least one cationic polymer is selected from the group consisting of chitosan, chitosan oligosaccharide, chitin, cyclodextrin, cationic gelatin, cationic dextran, cationic cellulose, polylysine, polyornithine, histone, collagen, chitosan-cysteine, chitosan-thiobutylamidine, chitosan-thioglycolic acid, and combinations thereof.

In some embodiments, the cationic polymer is chitosan present from about 0.05% to about 0.5%, by weight, based on the weight of the acne care composition.

In some embodiments, the acne care composition comprises at least one thickener.

In some embodiments, the at least one thickener is chosen from carrageenan, xanthan gum, sclerotium gum, or a combination thereof, and the at least one thickener is present in a range from about 0.1% to about 5%, by weight, based on the weight of the acne care composition.

In some embodiments, the at least one thickener is carrageenan present from about 0.5% to about 1.5%, by weight, based on the weight of the acne care composition.

In some embodiments, the acne care composition comprises at least one additive selected from the group consisting of anti-microbials, chelating agents, oils, fillers, penetrants, sequestrants, fragrances, dispersants, skin care actives, and combinations thereof.

In some embodiments, the acne care composition comprises at least one additive selected from the group consisting of sodium hydroxide, niacinamide, lactic acid, cetyl alcohol, trisodium ethylenediamine disuccinate, ceramides, and combinations thereof.

In some embodiments, the at least one surfactant comprises glyceryl stearate SE.

In some embodiments, the pH is from 4.7-5.9.

In another embodiment, the acne care composition comprises
a) surfactant comprising sodium cocoyl isethionate present at about 10% and glyceryl stearate SE present at about 5%;
b) at least one glycolipid present at about 3%;
c) at least one beta hydroxy acid comprising salicylic acid or a derivative thereof present at about 2%;
d) at least one clay comprising kaolin present at about 15%;
e) at least one cationic polymer comprising chitosan present at about 0.1%;
f) at least one thickener comprising one of carrageenan, xanthan gum, sclerotium gum, or a combination thereof present at about 1%;
g) at least one cosmetically acceptable solvent comprising water, and comprising glycerin present from about 10% to about 30%; and
h) at least one additive selected from the group consisting of sodium hydroxide, niacinamide, lactic acid, cetyl alcohol, trisodium ethylenediamine disuccinate, ceramides, and combinations thereof,
   all amounts by weight, based on the weight of the acne care composition,
   wherein the acne care composition demonstrates peak shear stress in a range from about 100 to about 1000 PA at shear rate in the range from 1 1/s-100 1/s, followed by a sharp drop in viscosity when shear stress is maintained.

In some embodiments, the at least one additive including sodium hydroxide, niacinamide, lactic acid, cetyl alcohol, trisodium ethylenediamine disuccinate, and ceramides.

In another embodiment, the disclosure provides a method for cleansing skin comprising applying to the skin of a subject an acne care composition, comprising:
a) surfactant comprising sodium cocoyl isethionate and glyceryl stearate SE;
b) at least one glycolipid;
c) at least one beta hydroxy acid comprising salicylic acid or a derivative thereof;
d) at least one clay comprising kaolin;
e) at least one cationic polymer comprising chitosan;
f) at least one thickener comprising one of carrageenan, xanthan gum, sclerotium gum, or a combination thereof;
g) at least one cosmetically acceptable solvent comprising water, and comprising glycerin; and
h) at least one additive selected from the group consisting of sodium hydroxide, niacinamide, lactic acid, cetyl alcohol, trisodium ethylenediamine disuccinate, ceramides, and combinations thereof,
   wherein the acne care composition demonstrates peak shear stress in a range from about 100 to about 1000 PA at shear rate in the range from 1 1/s-100 1/s, followed by a sharp drop in viscosity when shear stress is maintained.

In some embodiments of the method, the composition is one of (a) in the form of a spot treatment having a paste-like texture which is dotted onto skin and after a selected period of time rinsed off, (b) in the form of a cleanser having a paste-like texture which is applied to skin by rubbing and after a selected period of time rinsed off, and (c) and in the form of a mask having a paste-like texture which is applied to skin by rubbing and retained on the skin for a selected period of time then rinsed off.

Other features and advantages of the present invention will be apparent from the following more detailed description of the preferred embodiment which illustrates, by way of example, the principles of the invention.

### BRIEF DESCRIPTION OF THE DRAWINGS

FIG. 1 is a graph showing change in shear viscosity as a function of shear rate in exemplified compositions;

FIG. 2 is a graph showing change in shear stress as a function of shear rate in exemplified compositions; and

FIG. 3 is a graph showing change in shear stress as a function of shear rate in exemplified compositions.

It is to be understood that the foregoing and following descriptions are exemplary and explanatory only, and are not intended to be restrictive of any subject matter claimed.

### DETAILED DESCRIPTION

The disclosure relates to compositions for acne care and methods of using the compositions.

In various embodiments, the composition is a gentle surfactant based acne care product that may be provided in the form of one or more of a cleanser, mask, or leave on spot treatment, each intended for short term use and removal with at least water.

According to the invention, the acne care composition is provided in a versatile anti-acne product having a paste-like texture with a relatively thick consistency for leave on use. The acne care composition can be applied on acne spots without any dripping.

As a mask or facial cleanser, to cover large areas, the acne care composition demonstrates minimum resistance to spreading on the face, with good spreadability and remains in place without dripping. At low shear rate, the acne care composition behaves like yield stress fluid with a stable structure holding property, while at high shear rate, the shear stress exhibits an inflection point, followed by a sharp decrease in shear stress past peak while still at a high shear rate. Upon removal of sheer, the composition consistency returns to thick and paste like.

The examples show that the acne care composition demonstrates peak shear stress in a range from about 200 to about 1000 PA at shear rate in the range from 1 1/s-100 1/s, followed by a sharp drop in viscosity when shear stress is continued. Without being bound by theory, it is believed that at the peak inflection shear rate, there is a structural change in the micro aggregation of sodium cocoyl isethionate and associative ingredients that results in a release and thinning of the viscosity. Above inflection shear rate, lower viscosity results in better spreadability of the acne care composition.

### Compositions

### Surfactant:

In various embodiments, the acne care composition comprises the surfactant sodium cocoyl isethionate. In some embodiments, the acne care composition may include, or alternatively may be substantially free from, or exclude any one or more of nonionic, cationic, or anionic surfactants. In some embodiments, the acne care composition excludes all surfactants except sodium cocoyl isethionate.

In some embodiments, the composition also includes at least one additional surfactant selected from glyceryl oleate, glyceryl stearate, and cetyl alcohol. In some embodiments, the composition includes at least glyceryl stearate SE in addition to sodium cocoyl isethionate.

The concentration of sodium cocoyl isethionate present in the acne care composition is from about 1% to about 20%, or from about 2% to about 18%, or from about 5% to about 15%, or from about 8% to about 12%, or about 10%, or any suitable combination, sub-combination, range, or sub-range thereof by weight, based on the weight of the acne care composition. One of ordinary skill in the art, however, will appreciate that other ranges are within the scope of the invention.

Thus, sodium cocoyl isethionate is present, by weight, based on the total weight of the acne care composition, from about 1.0, 1.1, 1.2, 1.3, 1.4, 1.5, 1.6, 1.7, 1.8, 1.9, 2.0, 3, 4, 5, 6, 7, 8, 9, 10, 11, 12, 13, 14, 15, 16, 17, 18, 18 to about 20 weight percent, including increments and ranges therein and there between.

When present, an additional surfactant, for example, glyceryl stearate, is present in the acne care composition is from about 1% to about 20%, or from about 2% to about 18%, or from about 5% to about 15%, or from about 8% to about 12%, or from about 2% to about 6%, or about 5%, or any suitable combination, sub-combination, range, or sub-range thereof by weight, based on the weight of the acne care composition. One of ordinary skill in the art, however, will appreciate that other ranges are within the scope of the invention.

Thus, an additional surfactant, when present, is present, by weight, based on the total weight of the acne care composition, from about 1.0, 1.1, 1.2, 1.3, 1.4, 1.5, 1.6, 1.7, 1.8, 1.9, 2.0, 3, 4, 5, 6, 7, 8, 9, 10, 11, 12, 13, 14, 15, 16, 17, 18, 18 to about 20 weight percent, including increments and ranges therein and there between.

### Glycolipids:

The composition according to the invention comprises one or more glycolipids. In preferred embodiments, the at least one glycolipid is chosen from rhamnolipids, sophorolipids, or mixtures thereof.

In some embodiments the acne care composition includes only one glycolipid comprising rhamnolipid, for example sold under the name Rheance One™ by Evonik (INCI name: glycolipids), present from about 1% to about 4%, or from about 2% to about 3%, or about 3%.

The term “glycolipid” is understood as meaning a compound formed from a lipid to which are attached one or more sugar compounds.

The one or more glycolipids may be selected from rhamnolipids, sophorolipids, glucolipids, trehalolipids, cellobiose lipids, mannosylerythritol lipid, and mixtures thereof.

### Glucolipids:

The one or more glycolipids may be glucolipids, which contain a glucose moiety and can be represented by the general formula (I):

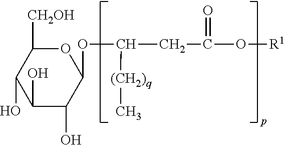

in which:
   R1 represents a hydrogen atom or a cation,
   p denotes an integer ranging from 1 to 4, and
   q denotes an integer ranging from 4 to 10, preferably equal to 6.

The glucolipids can be produced by the bacterium Alcaligenes sp. MM1.

The appropriate fermentation methods are reviewed by M. Schmidt in his doctoral thesis (1990), Technical University of Braunschweig, and by Schulz et al. (1991) Z. Naturforsch., 46C, 197-203. The glucolipids are recovered from the fermentation broth by solvent extraction using diethyl ether or a dichloromethane:methanol or chloroform:methanol mixture.

### Sophorolipids:

The one or more glycolipids may be sophorolipids, which contain a sophorose moiety and can be represented by the general formula (II):

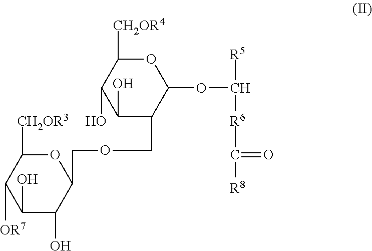

in which:
   R3 and R4 individually represent a hydrogen atom or an acetyl group,
   R5 represents a saturated or unsaturated, hydroxylated or non-hydroxylated hydrocarbon group having from 1 to 9 carbon atoms, preferably methyl,
   R6 represents a saturated or unsaturated, hydroxylated or non-hydroxylated hydrocarbon group having from 1 to 19 carbon atoms, with the proviso that the total number of carbon atoms in the groups R5 and R6 does not exceed 20 and is preferably from 14 to 18.

Sophorolipids may be incorporated into the composition according to the invention either in the form of the open-chain free acid, where R7 represents a hydrogen atom and R8 represents a hydroxy group OH, or in its lactone form, where a lactone ring is formed between R7 and R8, as indicated by formula (III):

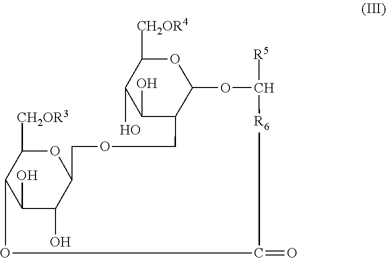

in which:
   R3, R4, R5 and R6 are as defined above,
with the proviso that at least one of R3 and R4 represents an acetyl group.

The sophorolipids can be produced by yeast cells, for example Torulopsis apicola and Torulopsis bombicola cells. The fermentation process generally uses sugars and alkanes as substrates.

Appropriate fermentation methods are reviewed in A. P. Tulloch, J. F. T. Spencer and P. A. J. Gorin, Can. J. Chem. (1962), 40, 1326, and U. Gobbert, S. Lang and F. Wagner, Biotechnology Letters (1984), 6 (4), 225. The resulting product is a mixture of various open-chain sophorolipids and of sophorolipid lactones that may be used in the form of mixtures, or the required form may be isolated.

It is possible to use as sophorolipids for example that sold under the Sopholiance S name by Givaudan and that sold under the BioToLife name by BASF.

### Trehalolipids:

The one or more glycolipids may be trehalolipids, which contain a trehalose fragment and can be represented by the general formula (IV):

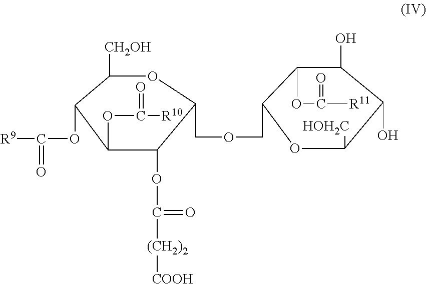

in which:
   R9, R10 and R11 individually represent a saturated or unsaturated, hydroxylated or non-hydroxylated hydrocarbon radical having from 5 to 13 carbon atoms.

The trehalolipids can be produced by bacterial fermentation using the marine bacterium Arthrobacter sp. Ek 1 or the freshwater bacterium Rhodococcus erythropolis. Appropriate fermentation methods are provided by Ishigami et al. (1987), J. Jpn. Oil Chem. Soc., 36, 847-851, Schultz et al. (1991), Z. Naturforsch., 46C, 197-203, and Passeri et al. (1991), Z. Naturforsch., 46C, 204-209.

### Cellobiose Lipids:

The one or more glycolipids may be cellobiose lipids, which contain a cellobiose fragment and can be represented by the general formula (V):

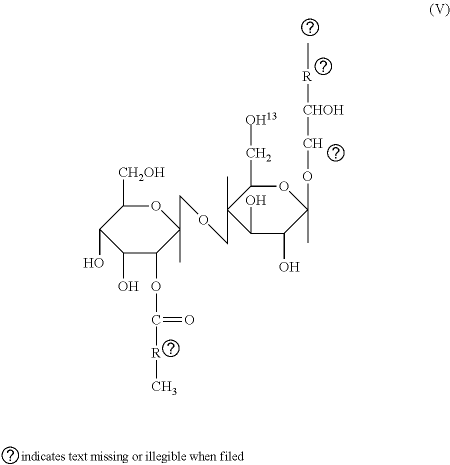

in which:
   R1 represents a hydrogen atom or a cation,
      R12 represents a saturated or unsaturated, hydroxylated or non-hydroxylated hydrocarbon radical having from 9 to 15 carbon atoms, preferably 13 carbon atoms,
      R13 represents a hydrogen atom or a acetyl group;
      R14 represents a saturated or unsaturated, hydroxylated or non-hydroxylated hydrocarbon radical having from 4 to 16 carbon atoms.

The cellobiose lipids can be produced by cells of fungi of the genus Ustilago. Appropriate fermentation processes are provided by Frautz, Lang and Wagner (1986), Biotech. Letts., 8, 757-762.

### Rhamnolipids:

The one or more glycolipids may be rhamnolipids.

The composition according to the invention preferably comprises one or more rhamnolipids.

Rhamnolipids are glycolipids produced by various bacterial species. They consist of one rhamnose fragment (mono-rhamnolipid) or of two rhamnose fragments (di-rhamnolipid) linked by a glycosidic bond to one, two or three chains of β-hydroxylated fatty acids linked to one another by an ester bond.

More specifically, these mono-rhamnolipids and di-rhamnolipids correspond to the following formula (VI):

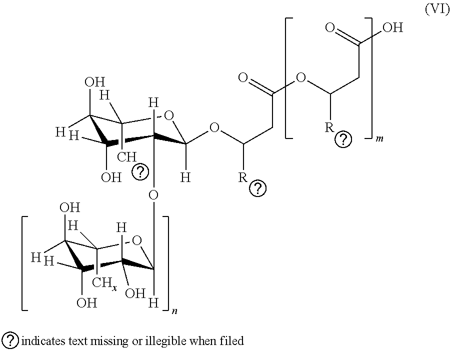

in which:
   m denotes an integer equal to 2, 1 or 0,
   n denotes an integer equal to 1 or 0, and
   R1 and R2, each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical.

Thus, when n is equal to 0, the formula (VI) protects mono-rhamnolipids and, when n is equal to 1, it protects di-rhamnolipids.

The composition according to the invention preferably comprises at least one di-rhamnolipid.

The composition according to the invention preferably comprises at least one di-rhamnolipid of formula (VI) in which:
m denotes an integer equal to 2, 1 or 0;
n denotes an integer equal to 1; and
R1 and R2, each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical, and also the salts thereof, solvates thereof and optical isomers thereof.

The glycosidic bond between the two rhamnose fragments may be in the alpha or beta configuration and is preferably in the alpha configuration.

In the context of the invention,
the salts of the di-rhamnolipids of formula (VI) are more particularly the carboxylate salts thereof with an organic or inorganic cation and especially with a cation selected from sodium, potassium, calcium and ammonium.
the solvated forms of the di-rhamnolipids of formula (VI) are more particularly those solvated with one or more molecules of water or of organic solvents, for example a hydrate or a solvate of a linear or branched alcohol, such as ethanol or isopropanol, the optically active carbon atoms of the fatty acids preferably being in the form of the R enantiomers, and
the term “alkyl” radical denotes a saturated, linear or branched aliphatic group; for example, a C1-C20 alkyl group having a linear or branched hydrocarbon chain of 1 to 20 carbon atoms, more particularly a methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl or eicosyl.

The composition according to the invention preferably comprises at least one di-rhamnolipid of formula (VI) in which:
m denotes an integer equal to 2, 1 or 0;
n denotes an integer equal to 1; and
R1 and R2, which are identical or different, are selected from pentenyl, hexenyl, heptenyl, octenyl, nonenyl, decenyl, undecenyl, dodecenyl and tridecenyl radicals and radicals of formula —(CH2)oCH3, with o denoting an integer ranging from 1 to 23, in particular from 3 to 15 and more particularly from 4 to 12.

According to one embodiment of the invention, the composition according to the invention comprises at least one di-rhamnolipids of general formula (VI) in which m is equal to 1

According to one embodiment of the invention, the composition according to the invention comprises a mixture of at least two, preferably at least three, di-rhamnolipids of general formula (VI) in which m is preferably equal to 1.

According to another embodiment of the invention, the composition according to the invention comprises a mixture comprising at least one mono-rhamnolipid.

More preferably, the composition according to the invention comprises at least one dirhamnolipid of the following formula (VII):

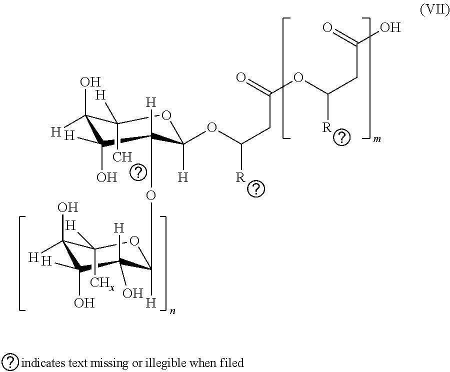

in which:
   m denotes an integer equal to 2, 1 or 0; preferably, m is equal to 1,
   n denotes an integer equal to 1,
   R1 is a —(CH2)p-CH3 radical, with p being an integer varying from 1 to 23, preferably from 4 to 12,
   R2 is a —(CH2)q-CH3 radical, with q being an integer varying from 1 to 23, preferably from 4 to 12, and also the salts thereof, solvates thereof and optical isomers thereof.

By way of illustration and without limiting the di-rhamnolipids of formula (VII) that may be suitable for the invention, mention may be made in particular of the compounds of formula di-RL-CXCY, such as are defined in Table 1 below.

The formula di-RL-CXCY is an alternative way of writing in order to represent a di-rhamnolipid (di-RL) functionalized by two radicals R1 and R2 respectively represented by the symbols CX and CY, the integers X and Y being respectively equal to p+4 and q+4.

TABLE 1
di-rhamnolipids of Formula (VII)

| Composés | Di-RL-CXCY | p | q |
|---|---|---|---|
| 1 | diRL-C8C8 | 4 | 4 |
| 2 | diRL-C8C10 | 4 | 6 |
| 3 | diRL-C10C8 | 6 | 4 |
| 4 | diRL-C10C10 | 6 | 6 |
| 5 | diRL-C10C12 | 6 | 8 |
| 6 | diRL-C12C10 | 8 | 6 |
| 7 | diRL-C12C12 | 8 | 8 |
| 8 | diRL-C12C14 | 8 | 10 |
| 9 | diRL-C14C12 | 10 | 8 |
| 10 | diRL-C14C14 | 10 | 10 |
| 11 | diRL-C14C16 | 10 | 12 |
| 12 | diRL-C16C14 | 12 | 10 |
| 13 | diRL-C16C16 | 12 | 12 |

According to a preferred embodiment, the composition according to the invention comprises at least one di-rhamnolipid of formula (VII) in which p and q are identical and equal to 6 and m is equal to 1, also referred to as di-RL-C10C10, or one of the salts, solvates and optical isomers thereof.

Preferably, the di-rhamnolipid of formula (VII) in which p and q are identical and equal to 6 and m is equal to 1 is present in the composition according to the invention in a proportion of at least 50% by weight and preferably of from 51% to 85% by weight, relative to the total weight of rhamnolipids.

According to another embodiment, the composition according to the invention comprises at least one di-rhamnolipid of formula (VII) in which m is equal to 1, p is equal to 6 and q is equal to 8.

According to another embodiment, the composition according to the invention comprises at least one di-rhamnolipid of formula (VI) in which n and m are equal to 1, R1 represents a —(CH2) oCH3 radical, with o being an integer varying from 4 to 12, and R2 is selected from the pentenyl, hexenyl, heptenyl, octenyl, nonenyl, decenyl, undecenyl, dodecenyl and tridecenyl radicals; preferably, R1 represents a —(CH2) 6CH3 radical and R2 a nonenyl radical.

According to another preferred embodiment, the composition according to the invention comprises a mixture of at least two, in particular at least three, di-rhamnolipids of formula (VI) or of formula (VII) selected from:
a di-rhamnolipid of formula (VII) in which p and q are identical and equal to 6 and m is equal to 1;
a di-rhamnolipid of formula (VII) in which m is equal to 1, p is equal to 6 and q is equal to 8; and
at least one di-rhamnolipid of formula (VI) in which n and m are equal to 1, R1 represents a —(CH2) oCH3 radical, with o being an integer varying from 4 to 12, and R2 is selected from the pentenyl, hexenyl, heptenyl, octenyl, nonenyl, decenyl, undecenyl, dodecenyl and tridecenyl radicals; preferably, R1 represents a —(CH2) 6CH3 radical and R2 a nonenyl radical.

Preferably, the composition according to the invention comprises a mixture of at least two, in particular at least three, di-rhamnolipids of formula (VI) or of formula (VII) selected from:
at least 50% by weight and preferably of from 51% to 85% by weight of a di-rhamnolipid of formula (VII) in which p and q are identical and equal to 6 and m is equal to 1, relative to the total weight of rhamnolipids.
from 0.5% to 25% by weight, preferably from 5% to 15% by weight, of a dirhamnolipid of formula (VII) in which p is equal to 6, q is equal to 8 and m is equal to 1, relative to the total weight of rhamnolipids, and
from 0.5% to 15% by weight, preferably from 3% to 12% by weight, preferably from 5% to 10% by weight, of a dirhamnolipid of formula (VI) in which n and m are equal to 1, R1 represents a —(CH2) 6CH3 radical and R2 represents a nonenyl radical, relative to the total weight of rhamnolipids.

As specified above, rhamnolipids are customarily prepared by processes known to those skilled in the art starting from bacterial producers, such as Pseudomonas.

Appropriate fermentation methods are reviewed by D. Haferburg, R. Hommel, R. Claus and H. P. Kleber in Adv. Biochem. Ing./Biotechnol. (1986), 33, 53-90, and by F. Wagner, H. Bock and A. Kretschmar in Fermentation (ed. R. M. Lafferty) (1981), 181-192, Springer Verlag, Vienna.

Use may be made, as rhamnolipid, of the one sold under the name Rheance One by Evonik (INCI name: glycolipids).

In some embodiments, the concentration of the at least one glycolipid is from about 0.1% to about 5%, or from about 1% to about 4%, or from about 2% to about 3%, or about 3%, or any suitable combination, sub-combination, range, or sub-range thereof by weight, based on the weight of the acne care composition. One of ordinary skill in the art, however, will appreciate that other ranges are within the scope of the invention.

Thus, the at least one glycolipid is present, by weight, based on the total weight of the acne care composition, from about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 to about 5 weight percent, including increments and ranges therein and there between.

### Beta Hydroxy Acid/Salicylic Acid:

The acne care composition comprises at least one beta hydroxy acid. In some particular embodiments, the acne care composition may include a beta hydroxy acid comprising salicylic acid.

In some particular embodiments, the acne care composition includes only one beta hydroxy acid comprising salicylic acid present from about 0.1% to about 2%, by weight of the acne care composition.

According to those embodiments that include salicylic acid, the composition according to the present invention comprises (b) at least one salicylic acid derivative according to formula (I). A single type of the salicylic acid derivative may be used, but two or more different types of the salicylic acid derivatives may be used in combination.

The salicylic acid derivatives in accordance with the present invention correspond to formula (I):

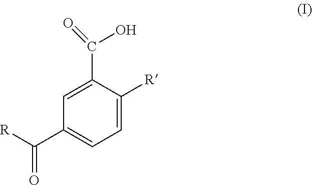

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

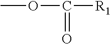

in which R1 denotes a linear or branched, saturated or unsaturated aliphatic chain containing 1 to 18 carbon atoms;
and also salts thereof derived from an inorganic or organic base.

Preferably, the R radical in formula (I) denotes a linear, branched or cyclic, saturated aliphatic chain containing from 3 to 11 carbon atoms; an unsaturated chain containing from 3 to 17 carbon atoms and comprising one or more conjugated or unconjugated double bonds; it being possible for said hydrocarbon-based chains to be substituted with one or more substituents, which may be identical or different, chosen from halogen atoms, a trifluoromethyl group, hydroxyl groups in free form or esterified with an acid containing from 1 to 6 carbon atoms, or a carboxyl function in free form or esterified with a lower alcohol containing from 1 to 6 carbon atoms.

In one preferred embodiment, the R radical in formula (I) denotes a linear or branched alkyl or alkenyl group, preferably alkyl group, containing 2 or more, preferably 3 or more, more preferably 4 or more, even more preferably 5 or more, and in particular 6 or more carbon atoms, and/or 22 or less, preferably 18 or less, even more preferably 14 or less, preferentially 12 or less, and in particular 10 or less carbon atoms.

Preferentially, R′ in formula (I) is a hydroxyl group or an ester group of formula:

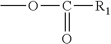

in which R1 denotes a radical-(CH2) n-CH3 where n is a number ranging from 0 to 14.

The salicylic acid derivatives that are more particularly preferred are those according to formula (I) in which the R radical is a C3-C10 alkyl group and/or R′ denotes hydroxyl.

Other particularly advantageous compounds are those in which R represents a chain derived from caprylic, linoleic, linolenic or oleic acid.

Another group of particularly preferred salicylic acid derivatives is constituted of compounds in which the R radical denotes a C3-C10 alkyl group bearing a carboxyl function in free form or esterified with a lower alcohol containing from 1 to 6 carbon atoms and R′ denotes hydroxyl.

The salicylic acid derivatives of formula (I) that may be used according to the present invention are in particular described in patents U.S. Pat. Nos. 6,159,479 and 5,558,871, FR 2,581,542, U.S. Pat. No. 4,767,750, EP 378 936, U.S. Pat. Nos. 5,267,407, 5,667,789, 5,580,549 and EP-A-570,230.

Among the particularly preferred salicylic acid derivatives of formula (I), mention may be made of 5-n-octanoylsalicylic acid (or capryloyl salicylic acid); 5-n-decanoylsalicylic acid; 5-n-dodecanoylsalicylic acid; 5-n-heptyloxysalicylic acid, and the corresponding salts thereof. The derivative in question is preferably 5-n-octanoylsalicylic acid.

For the purposes of the present invention, the salts of the salicylic acid derivatives are also considered. As the salts derived from inorganic bases, mention may particularly be made of those derived from alkali metal or alkaline-earth metal hydroxylated bases, for instance sodium hydroxide or potassium hydroxide, and ammonia. As regards the salts derived from the organic bases, mention may particularly be made of those derived from bases of amine or alkanolamine type.

More generally, the term “beta-hydroxy acid” is understood to mean, according to the present invention, a carboxylic acid having a hydroxyl functional group and a carboxylic functional group separated by two carbon atoms. A beta hydroxy acid can be present in the acne care composition in the form of the free acid and/or in the form of one of its associated salts (salts with an organic base or an alkali metal, in particular), especially according to the final pH imposed on acne care composition.

Suitable beta hydroxy acids include salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid, and combinations of these.

The acne care composition includes a concentration of beta hydroxy acid in a range from about 0.01% up to and not more than about 2% of beta hydroxy acid, by weight, based on the total weight of acne care composition. In some embodiments, the acne care composition may include up to and not more than about 2%, or about 1.9% of beta hydroxy acid. In some embodiments, the acne care composition may include up to and not more than about 1% of beta hydroxy acid. In some embodiments, the amount of beta hydroxy acid, if present is not more than about 0.40% to about 0.50%.

In some embodiments, the acne care composition may include from about 0.1% to about 2% of beta hydroxy acid, or any suitable combination, sub-combination, range, or sub-range thereof by weight, based on the weight of the acne care composition. One of ordinary skill in the art, however, will appreciate that other ranges are within the scope of the invention.

Thus, the at least one beta hydroxy acid is present in the acne care composition from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, to about 2.0%, by weight, based on the weight of the acne care composition, including increments and all ranges and subranges therein and there between.

### Clay:

In some embodiments the acne care composition may include at least one clay.

In some particular embodiments, the at least one clay is kaolin present from about 5% to about 20%, by weight of the acne care composition.

In some embodiments, if present, the at least one clay may be selected from the group of clays consisting of kaolinites, smectite, sodium smectite, calcium smectite, illite, chlorite, vermiculite, attapulgite, and combinations thereof. In some particular embodiments, the at least one clay may be kaolin. In some particular embodiments, the at least one clay may be a smectite clay in the mica family of phylosilicates. And in some particular embodiments, the at least one clay may comprise hectorite.

In some embodiments, the at least one clay is present in an amount from about 0.1% to about 20%, or from about 10% to about 18%, or about 15%, or any suitable combination, sub-combination, range, or sub-range thereof by weight, based on the weight of the acne care composition.

Thus, when at least one clay is present in the acne care composition it may be present by weight, based on the total weight of the acne care composition, from about 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, to about 20 percent, including increments and ranges therein and there between.

### Cosmetically Acceptable Solvent:

Compositions according to the disclosure contain at least one cosmetically acceptable solvent. In various embodiments, the cosmetically acceptable solvent may be chosen from water.

### Water:

In accordance with the various embodiments, water is present in the acne care composition in a range from about 10% to about 60%, or from about 20% to about 55%, or from about 25% to about 50%, or from about 28% to about 49%, or any suitable combination, sub-combination, range, or sub-range thereof by weight, based on the weight of the acne care composition. One of ordinary skill in the art, however, will appreciate that other ranges are within the scope of the invention.

Thus, water may be present by weight, based on the weight of the acne care composition, from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to about 60 weight percent, including increments and ranges therein and there between.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-saunier, water from Eaux Bonnes, water from Rochefort, water from Saint Christau, water from Les Fumades, water from Tercis-les-Bains or water from Avene.

The water phase may also comprise reconstituted thermal water, that is to say a water comprising trace elements such as zinc, copper, magnesium, etc., reconstituting the characteristics of a thermal water.

The acne care composition has a pH 4.7-5.9.

The pH may be adjusted to the desired value by addition of a base (organic or inorganic), for example sodium hydroxide, potassium hydroxide, or another suitable base, or combinations thereof.

### Water-Soluble Solvents:

In accordance with some embodiments, the acne care composition includes at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions.

In some particular embodiments, the water-soluble solvent includes glycerin present from about 5% to about 30%, or from about 10% to about 30%, by weight of the acne care composition.

As examples of organic solvents, non-limiting mentions can be made of ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols, glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, 1,2-hexanediol, 1,2-pentanediol, and 4-methyl-1,2-pentanediol.

Further non-limiting examples of water-soluble solvents include alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In accordance with the various embodiments the amount of the at least one water-soluble solvent is from about 0.1% to about 25%, or from about 0.1% to about 2%, or from about 0.1% to about 1%, or from about 0.1% to about 0.8%, or from about 0.1% to about 0.5%, or from about 1% to about 20%, or from about 1% to about 10%, or from about 2% to about 8%, or any suitable combination, sub-combination, range, or sub-range thereof by weight, based on the weight of the acne care composition. One of ordinary skill in the art, however, will appreciate that other ranges are within the scope of the invention.

In some embodiments, the acne care composition includes more than one water soluble solvent, each water soluble solvent present in an amount as set forth herein above, wherein each different water soluble solvent may be present within one of the ranges selected from the ranges set forth herein above.

Thus, water-soluble solvents may be present by weight, based on the total weight of the acne care composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

### Thickener:

In various embodiments, the acne care composition may comprise at least one stabilizer and/or thickening agent (thickener). The thickener may be thickening polymers. Non-limiting examples of thickening polymers include sclerotium gum, xanthan gum, carrageenan, acacia, agar, algin, alginic acid, ammonium alginate, amylopectin, calcium alginate, sodium alginate, calcium carrageenan, carnitine, carrageenan, dextrin, gelatin, gellan gum, guar gum, tragacanth gum, acacia gum, Arabic gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sodium carboxymethyl dextran, sodium carrageenan, tragacanth gum, xanthan gum, modified xanthan gum, biosacharide gum, chitin, levan, elsinan, collagen, gelatin, zein, gluten, soy protein, casein, and mixtures thereof.

According to some embodiments, the acne care composition comprises one or more of carrageenan, xanthan gum, and sclerotium gum, or a combination thereof, present in a range from about 0.1% to about 5%, or at about 1%, by weight of the acne care composition.

According to some embodiments, the acne care composition comprises carrageenan as a particularly preferred stabilizer/thickening agent, present at about 1%, by weight of the acne care composition.

The at least one thickener, when present, is present in the acne care composition from about 0.1% to about 5%, or from about 0.2% to about 3%, or from about 0.5% to about 2%, or about 1%, or any suitable combination, sub-combination, range, or sub-range thereof by weight, based on the weight of the acne care composition. One of ordinary skill in the art, however, will appreciate that other ranges are within the scope of the invention.

Thus, the at least one thickener may be present, by weight, based on the total weight of the acne care composition, from about 0.05, 0.06, 0.07, 0.08, 0.90, 0.1, 0.2, 0.3, 0.4, 0.5, 0.6, 0.7, 0.8, 0.9, 1.0, 2, 3, 4 to about 5 weight percent, including increments and ranges therein and there between.

### Cationic Polymer:

In various embodiments, the acne care composition may comprise at least one cationic polymer selected from nature-based polymers that are polysaccharides, and other natural (i.e., plant, animal, or bacterial based), synthetic, or modified cationic nature-based or synthetic polymers.

In some embodiments, the cationic polymer is a nature-based polymer. In general, a cationic nature-based polymer may be selected from cationic forms and cationic derivatives of polysaccharides isolated from algae, polysaccharides produced by microorganisms, and polysaccharides from higher plants, such as homogeneous polysaccharides.

In some embodiments, the acne care composition comprises the at least one nature-based cationic polymer comprising chitosan, where in some embodiments chitosan is selected from chitosan oligosaccharide, chitosan (or polymeric chitosan having molecular weight (MW) in a range from about 1 kDa to about 1000 kDa), derivatives of chitosan, including derivatives having enhanced solubility, or combinations thereof.

In various embodiments, chitosan has a molecular weight (MW) in a range from about 1 kDa to about 1000 kDa. In some particular embodiments, chitosan has a MW that is “low” and is in the range from about 1 kDa to about 20 kDa, or from about 10 kDa to about 20 kDa, or from about 12 kDa to about 18 kDa. In some embodiments, the chitosan has a Chitosan MW=˜ 27 kDa.

According to some embodiments, the acne care composition comprises chitosan present at about 0.1%, by weight of the acne care composition.

More generally, cationic nature-based polymer selected from polysaccharides may be chosen from polysaccharides that include chitosan, chitin, starches, alginates, celluloses, galactomannans such as guar gums, particularly cationic derivatives thereof, and combinations thereof. In some embodiments, the at least one cationic nature-based polymer selected from polysaccharides may be chosen from chitosan, chitosan derivatives, chitin, starch, starch derivatives, cellulose (for example, but not limited to, ethylcellulose, nitrocellulose, hemicellulose, and hemicellulose derivatives), alginates, including but not limited to, sodium alginate, and combinations thereof.

In some embodiments, the cationic polymer is a nature-based cationic polymer selected from: chitosan, chitosan oligosaccharide, polymeric chitosan having MW in a range from 1 kDa to about 1000 kDa, derivatives of chitosan, derivatives of chitosan having enhanced solubility, cyclodextrin, cationic gelatin, cationic dextran, cationic cellulose, polylysine, polyornithine, histone, collagen, chitosan-cysteine, chitosan-thiobutylamidine, chitosan-thioglycolic acid, or combinations thereof. In preferred embodiments, the cationic polymer is selected from chitosan, polylysine, or combinations thereof.

Examples of cationic polymers include polysaccharide-based delivery molecules (e.g., chitosan, cyclodextrin, cationic gelatin, cationic dextran, cationic cellulose), cationic peptides and their derivatives (e.g., polylysine, polyornithine), peptide/protein polymers ((e.g., histone, collagen), linear or branched synthetic polymers (e.g., polybrene, polyethyleneimine), natural polymers (e.g., histone, collagen), synthetic dendrimers, cationic thiolated biopolymers (nature-based thiomers or nature-based dendrimers, e.g., chitosan-cysteine, chitosan-thiobutylamidine as well as chitosan-thioglycolic acid).

Examples of nature-based cationic polymers include polysaccharide-based delivery molecules (e.g., chitosan, cyclodextrin, cationic gelatin, cationic dextran, cationic cellulose); cationic peptides and their derivatives (e.g., polylysine, polyornithine), peptide/protein polymers (e.g., histone, collagen); cationic thiolated biopolymers (nature-based thiomers or nature-based dendrimers, e.g., chitosan-cysteine, chitosan-thiobutylamidine, chitosan-thioglycolic acid); or combinations thereof.

According to the present invention, the cationic polymer may be selected from polylysines. A single type of polylysines may be used, or two or more different types of polylysines may be used in combination.

Polylysines correspond to the condensation of several amino acids of lysine.

Polylysine can be a natural homopolymer of L-lysine that can be produced by bacterial fermentation. Polylysines are typically used as a natural preservative in food products. Polylysine is a polyelectrolyte which is soluble in polar solvents such as water.

Polylysine can be, for example, epsilon-polylysine (or referred as “ε-polylysine”), which is a condensation of amino groups at the ε-position and carboxyl groups of lysines, or alpha-polylysine (or referred as “α-polylysine”), which is a condensation of amino groups at the α-position and carboxyl groups of lysines. Polylysine is commercially available in various forms, such as poly D-lysine and poly L-lysine. The polylysine is generally a condensate of L-lysines, i.e., poly L-lysine.

As an example of polylysine, mention may be made of:
Epsilon-poly-L-lysine of JNC CORPORATION which is a 25% solution of Epsilon-poly-L-lysine having a molecular weight of around 4,700 in aqueous solution.

The polylysine may be in the form of organic or inorganic salts. The addition salts with an acid are, for example, the hydrochloric or hydrobromic acid, sulfuric acid, citric acid, succinic acid, tartaric acid, lactic acid, para-toluenesulphonic acid, phosphoric acid, or acetic acid salts; or fatty acid salts, such as linoleic acid, oleic acid, palmitic acid, stearic acid, behenic acid, and 18-methylicosanoic acid. The addition salts with a base are, for example, a sodium salt, a calcium salt, or a hydroxyalkylamine salt, for example, N-methylglucamine, aminopropane diol or triethanolamine.

In some embodiments of the present invention, polylysine is in the free form. The term “free form” here indicates that the polylysine is not covalently bound to any other compounds.

The at least one cationic polymer, when present, is present in the acne care composition from about 0.05% to about 2%, or from about 0.05% to about 1%, or from about 0.05% to about 0.5%, or about 0.1%, or any suitable combination, sub-combination, range, or sub-range thereof by weight, based on the weight of the acne care composition. One of ordinary skill in the art, however, will appreciate that other ranges are within the scope of the invention.

Thus, the at least one cationic polymer may be present, by weight, based on the total weight of the acne care composition, from about 0.05, 0.06, 0.07, 0.08, 0.90, 0.1, 0.2, 0.3, 0.4, 0.5, 0.6, 0.7, 0.8, 0.9, 1.0, to about 2 weight percent, including increments and ranges therein and there between.

In some embodiments, the acne care composition may include or exclude any other cationic polymer, including a nature-based polymer or synthetic polymer.

In some examples, the synthetic polymers are non-silicone based, and in some embodiments may be selected from the group consisting of polyquaterniums, that is polymers having quaternary ammonium centers in the polymer, for example, including polyquaternium-47 (1-Propanaminium, N,N,N-trimethyl-3-[(2-methyl-1-oxo-2-propenyl)amino]-, chloride, polymer with methyl 2-propenoate and 2-propenoic acid).

Other non-limiting examples of such polyquaternium compounds may be selected from diallyidimethylammonium chloride/acrylic acid copolymers sold under the names MERQUAT 280 POLYMER or MERQUAT 280NP POLYMER or MERQUAT 281 POLYMER or MERQUAT 295 POLYMER, by the company Nalco (Lubrizol) (INCI name: Polyquaternium-22); the copolymer of methacrylamidopropyltrimonium chloride, of acrylic acid and or methyl acrylate, sold under the name MERQUAT 2001 POLYMER OR MERQUAT 2001N POLYMER by the company Nalco (Lubrizol) (INCI name: Polyquaternium-47); the acrylamide/dimethyldiallylammonium chloride/acrylic acid terpolymer sold under the name MERQUAT 3330DRY POLYMER or MERQUAT 3330PR POLYMER or MERQUAT 3331PR POLYMER or MERQUAT 3940 POLYMER or MERQUAT PLUS 3330 POLYMER OR MERQUAT PLUS 3331 POLYMER by the company Nalco (Lubrizol) (INCI name:: Polyquaternium-39); an ampholytic terpolymer consisting of methacrylamidopropyl trimethyl ammonium chloride (MAPTAC), acrylamide and acrylic acid, sold under the name MERQUAT 2003PR POLYMER by the company Nalco (Lubrizol) (INCI name:: Polyquaternium-53).

Other non-limiting examples of such polyquaternium compounds may be selected from Polyquaternium-30, Polyquaternium-35, Polyquaternium-45, Polyquaternium-50, Polyquaternium-54; Polyquaternium-57; Polyquaternium-63; Polyquaternium-74; Polyquaternium-95;

Polyquaternium-76; Polyquaternium-86; Polyquaternium-89;

Polyquaternium-98, Polyquaternium-104; Polyquaternium-111; Polyquaternium-112, and mixtures thereof.

Synthetic polymer may include cationic acrylic polymers, for example, an amorphous functional acrylic polymer grafted onto a polyethylene backbone such as SYNTRAN™ 5330, which is a quaternary modified olefin grafted technology.

Synthetic polymers may be selected from, polyacrylates such as those identified in the International Cosmetic Ingredient Dictionary and Handbook (9 th ed. 2002) such as, for example, polyacrylate-1, polyacrylate-2, polyacrylate-3, polyacrylate-4, polyacrylate-16, polyacrylate-17, polyacrylate-18, polyacrylate-19, polyacrylate-21, and mixtures thereof.

### Optional Additives:

In some embodiments, there may be one or more optional actives or other ingredients (herein, “additives”) present in the acne care composition, the one or more additives optionally selected from: anti-microbials; chelating agents; oils; fillers; penetrants; sequestrants; fragrances; dispersants; skin care actives such as ceramides, for example, SODIUM LAUROYL LACTYLATE (and) CERAMIDE NP (and) CERAMIDE AP (and) PHYTOSPHINGOSINE (and) CHOLESTEROL (and) XANTHAN GUM (and) CARBOMER (and) CERAMIDE EOP; opacifiers; alpha-hydroxy acids; organic and inorganic UV filters; citric acid; phenylethyl resorcinol; hydroxypropyl tetrahydropyrantriol; hydroxyacetophenone; antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid; hyaluronic acid and its derivatives; escin (also known as Aescin, a mixture of saponins with anti-inflammatory, vasoconstrictor and vasoprotective effects found in Aesculus hippocastanum); retinol; niacinamide; and vitamins and vitamin derivatives, such as tocopherol and ascorbic acid; and combinations thereof.

Although the aforementioned optional additives are given as an example, it will be appreciated that other optional components compatible with cosmetic applications known in the art may be used. And of course, any one of the aforementioned additives may be excluded.

In some embodiments, the acne care composition includes at least one additive selected from the group consisting of sodium hydroxide, niacinamide, lactic acid, cetyl alcohol, trisodium ethylenediamine disuccinate, ceramides, and a combination thereof.

In some embodiments, the acne care composition includes additives comprising sodium hydroxide (0.3-0.5%), niacinamide (2%), lactic acid (0.05%), cetyl alcohol (2.5%), trisodium ethylenediamine disuccinate (0.5-1%), and ceramides (sodium lauroyl lactylate (and) ceramide NP (and) ceramide AP (and) phytosphingosine (and) cholesterol (and) xanthan gum (and) carbomer (and) ceramide EOP) (*final active concentration 0.25%)).

In accordance with the various embodiments, the amounts of additives, for example, actives and other components, that may be present in the acne care composition can range from about 0.001% to about 50%, or from about 0.5% to about 30%, or from about 1.5% to about 20%, and from about 5% to about 15%, or any suitable combination, sub-combination, range, or sub-range thereof by weight, based on the weight of the acne care composition.

Thus, one or a combination of additives may be present in the acne care composition, by weight, based on the weight of the acne care composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

### EXAMPLES

The following examples are intended to be non-limiting and explanatory in nature only. In the Examples, amounts are expressed in percentage by weight (wt %) of active materials, relative to the total weight of the composition.

### Example 1: Raw Materials

Percentages of each ingredient as may be exemplified in the following composition examples are shown as amount of raw materials, wherein the raw materials may be present in an amount that is equal to the amount of active, or if the raw material has a concentration of active that is less than 100%, then the cosmetic composition includes the raw material that includes active and a suitable solvent, wherein the concentration of active in the raw material is provided herein below in Table 2. When referring to the inventive and comparative examples, it should be presumed that each ingredient as shown in the exemplified composition is final amount of active unless otherwise indicated.

TABLE 2
Select Raw Materials (including RMs having
active concentrations of less than 100%)

| | | ~% Active |
|---|---|---|
| RM | Description | In Rm |
| Glycolipid | Rheance One (TM) | 50% |
| [Rhamnolipids] | | |
| Kaolin | Na/powder | |
| Ceramides | sodium lauroyl lactylate (and) ceramide NP | 1.55% |
| | (and) ceramide AP (and) phytosphingosine | |
| | (and) cholesterol (and) xanthan gum (and) | |
| | carbomer (and) ceramide EOP | |

### Example 2: Inventive and Comparative Compositions

Table 3 includes inventive compositions A and B.

TABLE 3
INVENTIVE COMPOSITIONS A-B

| INGREDIENT | INV A | INV B |
|---|---|---|
| GLYCERYL STEARATE SE | 5.00 | 5.00 |
| CARRAGEENAN | 1.00 | 1.00 |
| SALICYLIC ACID | 2.00 | 2.00 |
| WATER | 47.70 | 28.15 |
| GLYCERIN | 10.00 | 30.00 |
| KAOLIN | 15.00 | 15.00 |
| SODIUM COCOYL ISETHIONATE | 10.00 | 10.00 |
| sodium hydroxide, niacinamide, lactic acid, cetyl | 6.2 | 5.75 |
| alcohol, trisodium ethylenediamine disuccinate, | | |
| and ceramides (*final active concentration) | | |
| GLYCOLIPIDS (*final active concentration) | 3.00 | 3.00 |
| CHITOSAN | 0.10 | 0.10 |

The inventive and comparative compositions in Table 4, below, include the same ingredients as one of Inventive A or B but either lack or have a substituted ingredient.

TABLE 4
INVENTIVE COMPOSITIONS C-H and Comparative
Compositions 1 and 2

| | |
|---|---|
| INV C | Inv A - no thickener (replaces carrageenan) |
| INV D | Inv A - no thickener (replaces carrageenan) |
| INV E | Inv A -thickener is xanthan gum @1% (replaces carrageenan) |
| INV F | Inv B -thickener is xanthan gum @1% (replaces carrageenan) |
| INV G | Inv A -thickener is sclerotium gum @1% (replaces |
| | carrageenan) |
| INV H | Inv B -thickener is sclerotium gum @1% (replaces |
| | carrageenan) |
| COMP 1 | Inv A - substitute surfactant sodium cocoyl isethionate with |
| | sodium lauroyl methyl isethionate @10% |
| COMP 2 | Inv B - substitute surfactant sodium cocoyl isethionate with |
| | sodium lauroyl methyl isethionate @10% |

### Example 3: Characterization of Compositions

Samples of all compositions were prepared and evaluated for change in viscosity and shear stress when shear was applied to simulate rubbing on application to skin.

Sample viscosity flow profile was measured by rheometer in flow sweep mode between shear rate of 0.01 1/s and 1000 1/s. Prior to the measurement, sample was conditioned at 25 C, and allowed to perform preshear then equilibration.

### Results:

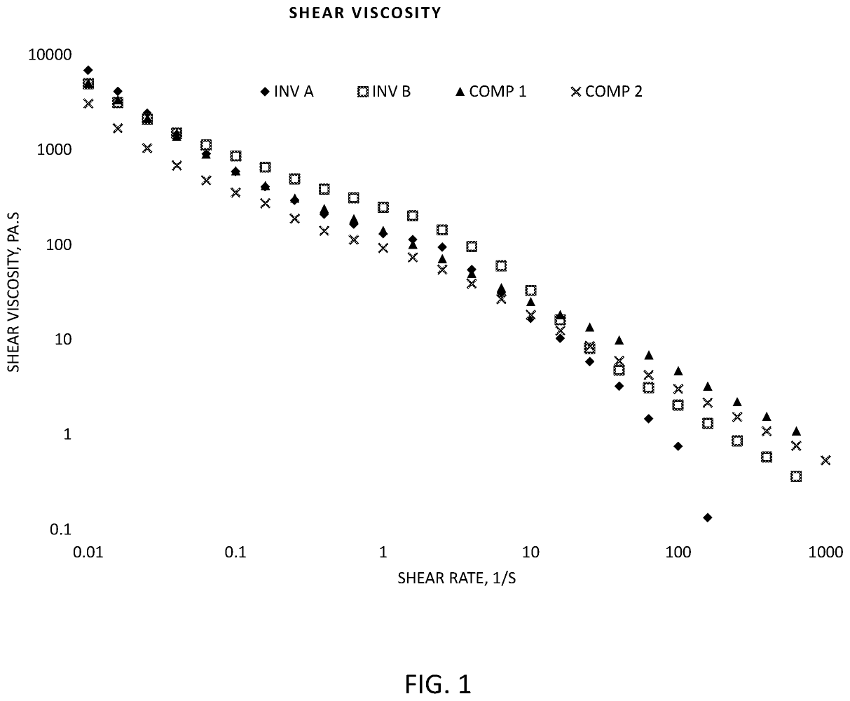

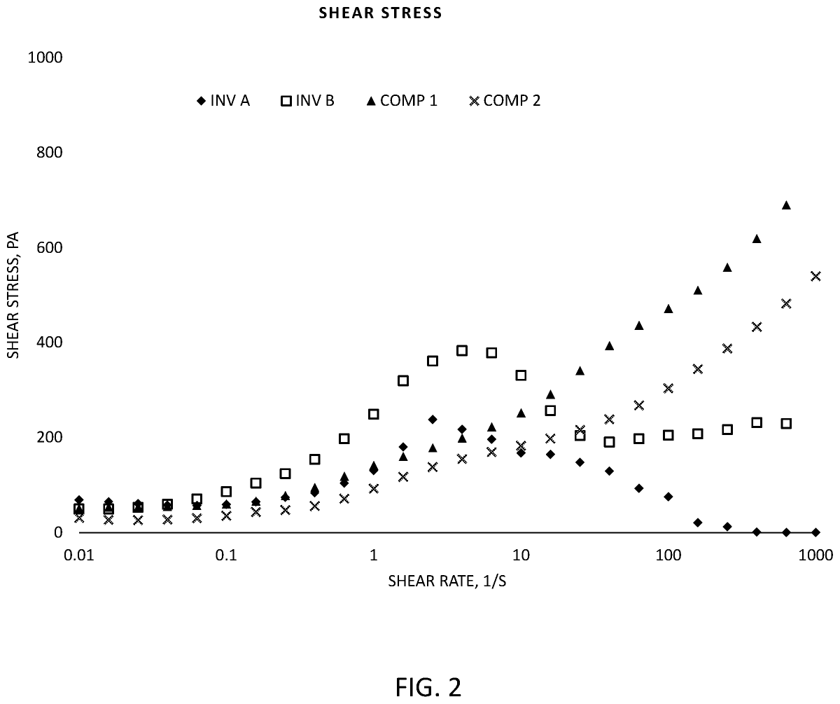

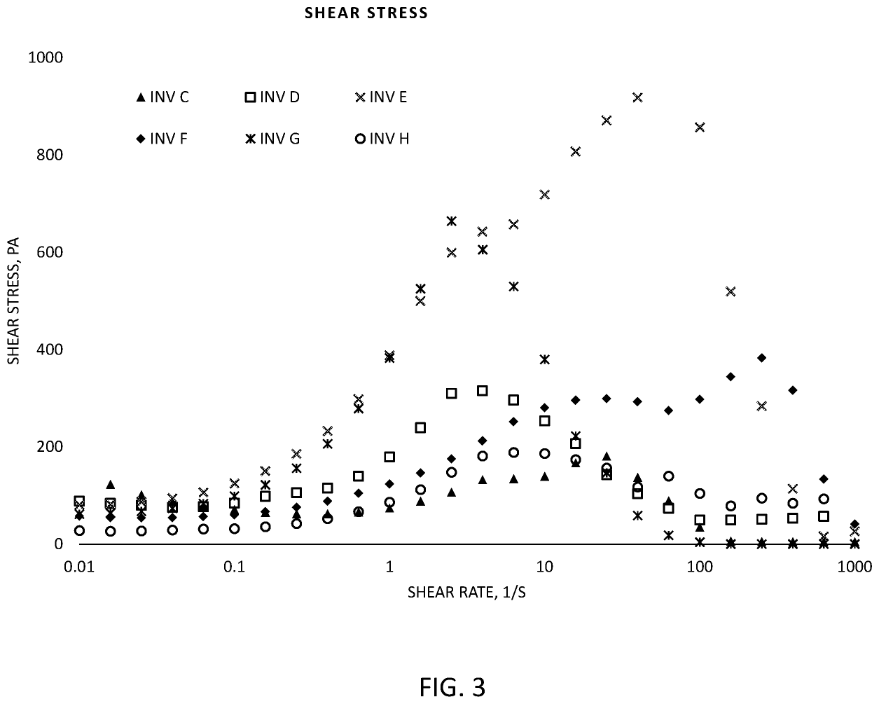

Referring to the drawings, FIG. 1 shows the change in viscosity in all tested compositions under study, while FIG. 2 and FIG. 3, respectively, show the behavior of the tested compositions when a high shear is applied.

As shown, all inventive and comparative compositions exhibit shear thinning behavior. However, in formulations with sodium cocoyl isethionate (Inventives A-H), viscosity shows more drastic reduction past peak shear stress vs. Comp 1 and Comp 2 with sodium methyl lauroyl isethionate. Indeed, in the comparatives which include surfactant sodium lauroyl methyl isethionate, shear stress keeps increasing with shear rate, and shear viscosity is simple shear thinning, no peak observed in shear stress.

The observed reaction to shear viscosity change is believed to relate to structure change in the formulation, which is evidenced by the peak shear stress at shear rate around 1-100 1/s. It is believed, without being bound by theory, that the sharp decrease in shear stress past peak indicates evolution of microstructure in the formulas. In contrast, in Comp 1 and Comp 2 with sodium methyl lauroyl isethionate, shear stress steadily goes up along with the rise of shear rate, which means gradual increase in resistance to shear but no structure change could be interpreted.

Presence of peak shear stress and drastic decrease in viscosity result in better spreadability of product.

Referring again to the drawings, the presence of peak shear stress is independent of polymer and concentration of glycerin, but relates to the presence of sodium cocoyl isethionate surfactant.

While the disclosure has been described with reference to a preferred embodiment, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

### Definitions and Terms

The transitional terms “comprising”, “consisting essentially of” and “consisting of”, when used in the appended claims, in original and amended form, define the claim scope with respect to what unrecited additional claim elements or steps, if any, are excluded from the scope of the claim(s). As used herein, the terms “comprising,” “having,” and “including” (or “comprise,” “have,” and “include”) are used in their open, non-limiting sense. The term “comprising” is intended to be inclusive or open-ended and does not exclude any additional, unrecited element, method, step, or material.

The term “consisting of” excludes any element, step, or material other than those specified in the claim and, in the latter instance, impurities ordinary associated with the specified material(s).

The term “consisting essentially of” limits the scope of a claim to the specified elements, steps, or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point.

A range given of “about 3% to 7%” is intended to have the term “about” modifying both the 3% and the 7% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient in the raw material except as specifically stated. All amounts given herein are relative to the amount of active material, unless otherwise indicated.

All percentages, parts and ratios herein are based upon the total weight of embodiments of the composition of the present disclosure, unless otherwise indicated.

As used herein, the term “surfactants,” as well as any specifically identified surfactants, includes salts of the surfactants even if not explicitly stated.

As used herein, the term “synthetic” means a material that is not of natural origin. The term “natural” and “naturally-sourced” and “nature-based” means a material of natural origin, such as derived from plants, which also cannot be subsequently chemically or physically modified. “Plant-based” means that the material came from a plant.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

As used herein, the term “substantially free” or “essentially free” as used herein means the specific material may be present in small amounts that do not materially affect the basic and novel characteristics of the embodiments of the composition according to the disclosure or the material may be absent. For instance, there may be less than 2% by weight of a specific material added to a composition, based on the total weight of the compositions (provided that an amount of less than 2% by weight does not materially affect the basic and novel characteristics of embodiments of the composition according to the disclosure. Similarly, the compositions may include less than 2%, less than 1.5%, less than 1%, less than 0.5%, less than 0.1%, less than 0.05%, or less than 0.01%, or none (0%) of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. An acne care composition, comprising:
a) at least one surfactant comprising sodium cocoyl isethionate;
b) at least one glycolipid;
c) at least one beta hydroxy acid;
d) at least one clay; and
e) at least one cosmetically acceptable solvent comprising water and glycerin,
wherein the acne care composition demonstrates peak shear stress in a range from about 100 to about 1000 PA at shear rate in the range from 1 1/s-100 1/s, followed by a sharp drop in viscosity when shear stress is maintained.

2. The composition of claim 1, wherein the at least one glycolipid is chosen from rhamnolipids, sophorolipids, glucolipids, trehalolipids, cellobiose lipids, mannosylerythritol lipid, or a mixture thereof.

3. The composition of claim 1, wherein the at least one glycolipid is chosen from rhamnolipids present from about 1% to about 4%, by weight, based on the weight of the acne care composition.

4. The composition of claim 1, wherein the at least one beta hydroxy acid comprises salicylic acid or a derivative thereof present from about 0.1% to about 2%, by weight, based on the weight of the acne care composition.

5. The composition of claim 1, wherein the at least one clay comprises kaolin present from about 5% to about 20%, by weight, based on the weight of the acne care composition.

6. The composition of claim 1, wherein glycerin is present from about 10% to about 30%, by weight, based on the weight of the acne care composition.

7. The composition of claim 1, comprising at least one cationic polymer.

8. The composition of claim 7, wherein the at least one cationic polymer is selected from the group consisting of chitosan, chitosan oligosaccharide, chitin, cyclodextrin, cationic gelatin, cationic dextran, cationic cellulose, polylysine, polyornithine, histone, collagen, chitosan-cysteine, chitosan-thiobutylamidine, chitosan-thioglycolic acid, and combinations thereof.

9. The composition of claim 7, wherein the cationic polymer is chitosan present from about 0.05% to about 0.5%, by weight, based on the weight of the acne care composition.

10. The composition of claim 1, comprising at least one thickener.

11. The composition of claim 10, wherein the at least one thickener is chosen from carrageenan, xanthan gum, sclerotium gum, or a combination thereof, and the at least one thickener is present in a range from about 0.1% to about 5%, by weight, based on the weight of the acne care composition.

12. The composition of claim 10, wherein the at least one thickener is carrageenan present from about 0.5% to about 1.5%, by weight, based on the weight of the acne care composition.

13. The composition of claim 1, comprising at least one additive selected from the group consisting of anti-microbials, chelating agents, oils, fillers, penetrants, sequestrants, fragrances, dispersants, skin care actives, and combinations thereof.

14. The composition of claim 1, comprising at least one additive selected from the group consisting of sodium hydroxide, niacinamide, lactic acid, cetyl alcohol, trisodium ethylenediamine disuccinate, ceramides, and combinations thereof.

15. The composition of claim 1, wherein the at least one surfactant comprises glyceryl stearate SE.

16. The composition of claim 1, wherein the pH is from 4.7-5.9.

17. An acne care composition, comprising:
a) surfactant comprising sodium cocoyl isethionate present at about 10% and glyceryl stearate SE present at about 5%;
b) at least one glycolipid present at about 3%;
c) at least one beta hydroxy acid comprising salicylic acid or a derivative thereof present at about 2%;
d) at least one clay comprising kaolin present at about 15%;
e) at least one cationic polymer comprising chitosan present at about 0.1%;
f) at least one thickener comprising one of carrageenan, xanthan gum, sclerotium gum, or a combination thereof present at about 1%;
g) at least one cosmetically acceptable solvent comprising water, and comprising glycerin present from about 10% to about 30%; and
h) at least one additive selected from the group consisting of sodium hydroxide, niacinamide, lactic acid, cetyl alcohol, trisodium ethylenediamine disuccinate, ceramides, and combinations thereof,
all amounts by weight, based on the weight of the acne care composition,
wherein the acne care composition demonstrates peak shear stress in a range from about 100 to about 1000 PA at shear rate in the range from 1 1/s-100 1/s, followed by a sharp drop in viscosity when shear stress is maintained.

18. The composition of claim 16, the at least one additive including sodium hydroxide, niacinamide, lactic acid, cetyl alcohol, trisodium ethylenediamine disuccinate, and ceramides.

19. A method for cleansing skin comprising applying to the skin of a subject an acne care composition, comprising:
a) surfactant comprising sodium cocoyl isethionate and glyceryl stearate SE;
b) at least one glycolipid;
c) at least one beta hydroxy acid comprising salicylic acid or a derivative thereof;
d) at least one clay comprising kaolin;
e) at least one cationic polymer comprising chitosan;
f) at least one thickener comprising one of carrageenan, xanthan gum, sclerotium gum, or a combination thereof;
g) at least one cosmetically acceptable solvent comprising water, and comprising glycerin; and
h) at least one additive selected from the group consisting of sodium hydroxide, niacinamide, lactic acid, cetyl alcohol, trisodium ethylenediamine disuccinate, ceramides, and combinations thereof,
wherein the acne care composition demonstrates peak shear stress in a range from about 100 to about 1000 PA at shear rate in the range from 1 1/s-100 1/s, followed by a sharp drop in viscosity when shear stress is maintained.

20. The method of claim 19, wherein the composition is one of (a) in the form of a spot treatment having a paste-like texture which is dotted onto skin and after a selected period of time rinsed off, (b) in the form of a cleanser having a paste-like texture which is applied to skin by rubbing and after a selected period of time rinsed off, and (c) and in the form of a mask having a paste-like texture which is applied to skin by rubbing and retained on the skin for a selected period of time then rinsed off.
